(19) United States
(12) Patent Application Publication
(10) Pub. No.: US 2005/0112145 A1
(43) Pub. Date: May 26, 2005

(54) Anthrax antigenic compositions

(76) Inventors: Michael John Hudson, Wiltshire (GB); Andrew Robinson, Wiltshire (GB); Nigel Silman, Wiltshire (GB); Bassam Hallis, Wiltshire (GB); Charles Penn, Wiltshire (GB)

Correspondence Address: STERNE, KESSLER, GOLDSTEIN & FOX PLLC, 1100 NEW YORK AVENUE, N.W., WASHINGTON, DC, 20005, US

(21) Appl. No.: 10/494,384

(22) PCT Filed: Nov. 1, 2002
(86) PCT No.: PCT/GB02/04985

(30) Foreign Application Priority Data
Nov. 1, 2001 (GB) 0126266.6

(51) Int. Cl. A61K 39/02

(52) U.S. Cl. 424/235.1

(57) ABSTRACT

An antigenic pharmaceutical composition is provided comprising Protective Antigen (PA) and Lethal Factor (LF), wherein said PA and/or LF lacks a functional binding site, thereby preventing said PA and LF from binding together via said binding site or thereby preventing said PA from binding to a native PA cell receptor via said binding site, and wherein said composition is substantially non-toxic to animal cells. The composition is for preventing or minimising anthrax toxicity in mammals, preferably in humans. Also provided are DNA and RNA based vaccines encoding the antigenic components of said pharmaceutical composition. The present specification also describes antibodies that bind to at least one of PA, LF or EF, which binding thereby prevents:—(i) PA from binding to LF or EF, or to a native PA cell receptor; or (ii) LF from binding to PA; or (iii) EF from binding to PA.

The present invention relates to antigenic compositions that provide protection against anthrax-associated toxicity, and to methods for preparing said compositions.

Anthrax vaccine has been manufactured by the present Applicant for over 40 years and, since 1979′, has been the subject of a UK Product Licence (PL1511/0037) held by the Secretary of State for Health. However, within that time there has been little product development or advance in its manufacturing process.

The above vaccine preparation is now described in more detail. Cultures of the toxigenic, non-capsulating B. anthracis 34F2 “Sterne” strain [see Sterne, M. (1939). Onderstepoort J. of Veterinary Science and Animal Industry, 13, pp. 307-312] are grown in multiple 500 ml volumes in a partially defined medium in Thompson bottles at 37° C. until the pH of selected culture bottles falls below pH 7.4.

At the end of the growth period (approximately 24-28 hours) the cultures are harvested by aspiration, and the pooled supernatant fluids sterilised by filtration. Potassium aluminium sulphate solution is added, and the resulting solution mixed. The pH is then adjusted to 5.8-6.2, and the resulting flocculent (‘alum-precipitation’) allowed to settle under gravity for up to one week at 5° C.

The precipitate is then concentrated 20-fold (by volume) by aspiration, and diluted 1:4 with a saline solution to provide a ‘5-fold’ concentrate of anthrax vaccine precipitate (AVP). This is the antigenic composition that is used for vaccine formulation. Although the vaccine is subjected to animal tests for potency and safety prior to human use, there is no separate routine biochemical characterisation.

One further cell-free anthrax vaccine is available for human use. This vaccine is produced in the United States of America and is broadly similar to that available under PL1511/0037, except that a different B. anthracis strain is used and grown anaerobically. The process is fermenter-based, and the culture filtrate is absorbed on to an aluminium hydroxide suspension.

Other available vaccines comprise live, attenuated spore suspensions. However, because of the inherent risks associated with attenuated pathogens, these vaccines are usually restricted to non-human use.

Anthrax toxin consists of the three distinct polypeptides known as protective antigen (PA), oedema factor (EF), and lethal factor (LF). The toxin components act in specific binary combinations of PA and EF to form oedema toxin (ET), which causes tissue oedema, and of PA and LF to form lethal toxin (LT), which is lethal to laboratory animals and causes lysis of monocyte and macrophage cells. Lethal toxin is considered to be the principal cause of anthrax-associated death as a consequence of its cytotoxic effects on peripheral macrophages and other cells.

PA acts as a target cell binding moiety and, after a site-specific N-terminal activation by a cell-associated protease, oligomerises and provides a high affinity binding component for which EF and LF compete. Following binding of EF or LF to activated PA, the resulting ET or LT complexes become internalised by an acidic endosome compartment, and the toxin factors EF and LF are thereby delivered into the cytosol of the target cell.

EF is a calcium- and calmodulin-dependent adenylyl cyclase that catalyses the conversion of intracellular ATP to cAMP. EF is active in a variety of intracellular signalling pathways, and is thereby capable of disrupting a range of cellular processes.

LF is a Zn²⁺-dependent metalloprotease that cleaves and inactivates the dual specificity mitogen-activated protein kinase kinases MAPKK/1 and 2, MEK-1 and MEK-2, and probably other proteins.

A survey of in vitro or in vivo published data on anthrax vaccines for human use indicates the following:—
1. to date, all effective anthrax vaccines contain or produce PA (ie. either the 83 kDa pro-form, or its activated 63 kDa derivative). In fact, the current dogma is that PA is necessary and sufficient alone to produce an effective anthrax vaccine, and efforts are underway to develop such a vaccine [see, for example, Baillie, L. (2001), 91, pp. 609-613];
2. the non-capsulated, toxigenic live-spore vaccines effect a higher degree of protection against all B. anthracis strains so far tested than do the licensed cell-free vaccines [see Little, S. F. (1986) Inf. and Imm., vol. 52, No. 2, pp. 509-512];
3. the current cell-free vaccines are generally, poorly defined and may vary significantly in effectiveness on a batch-by-batch basis. Accordingly, each batch must be individually tested for efficiacy in an animal model prior to human use;
4. the current cell-free anthrax vaccine manufacturing process is evaluated only on completion of the production process and packaging of the final product. Thus, in the event that any one batch of vaccine material should not meet the validation test criteria, the contributing factors can not be identified readily. Such factors may differ between manufactured batches and the lack of understanding exacerbates any difficulties encountered in the manufacturing process;
5. as a result of the poorly defined nature of current cell-free vaccines, these vaccines may contain quantities of PA together with LF and/or EF which, upon in vivo (or in vitro) activation of PA to the 63 kDa form, may form LT and ET and exert adverse effects on the recipient of the vaccine. Such vaccines may, of course, also contain other B. anthracis proteins, both secreted and lysis products, peptidoglycan, nucleic acid and carbohydrate, which may compromise protective efficiacy;
6. the current cell-free vaccine compositions are highly variable in terms of LF, PA, and EF concentrations, so much so that EF may be absent from some preparations; and
7. the current cell-free compositions are highly variable in terms of total protein content. Thus, the concentration of toxin components present in a given composition may vary significantly. This, in turn, may affect efficacy and potential toxicity in humans.
   Over the last few years there has been notable academic research in the anthrax field, which research has allowed identification of the native binding sites and translocation domain of PA [see Bhatnagas, R. (2001) Critical Rev. in Microbiol., 27(3), pp. 167-200; and Batra, S. (2001.) Biochem. and Biophys. Res. Comm., 281, pp. 186-192]. Thus, the structure and binding/translocation domains of PA have been well documented.

Similarly, academic based research has allowed elucidation of the binding and enzyme function domains of LF [see Bragg, T. S. (1989) Gene, 81, pp. 45-54; Quinn, C. P. (1991) J. Biol. Chem, vol. 266, No. 30, pp. 20124-20130; Gupta, P. (2001) Biochem. and Biophys. Res. Comm., 280, pp. 158-163; and Klimpel, K. R. (1994) Mol. Microbiol., 13(6), pp. 1093-1100]. Thus, the structure and binding/enzyme function domains of LF have been well documented.

Recently, a second generation “recombinant” anthrax vaccine has been proposed by The Ohio State University Research Foundation [see WO 01/45639; and Price, B. M. (2001) Inf. and Immun., vol. 69, No. 7, pp. 4509-4515]. The described vaccine is based on PA and LF, wherein the LF molecule has been modified so as to be zinc metalloprotease negative. Thus, the described PA and LF components are fully capable of binding to one another to form an LT molecule, but the resulting LT molecule is not cytotoxic as there is no active zinc metalloprotease function present with the LF component.

In view of the increasing threats of bioterrorism and biological warfare, there is a need for alternative anthrax vaccines, and for vaccines that address one or more of the above-identified problems.

Thus, according to a first aspect of the present invention, there is provided an antigenic composition for use as a vaccine, which composition comprises Protective Antigen (PA) and Lethal Factor (LF), wherein said PA and/or LF lacks a functional binding site, thereby preventing said PA and LF from binding together via said binding site or thereby preventing said PA from binding to a native PA cell receptor via said binding site, and wherein said composition is substantially non-toxic to animal cells.

Reference to PA throughout this specification embraces both the 83 and 63 kDa forms of PA.

In the context of the present invention, PA lacks a functional binding site if it is incapable of binding to either the native target cell receptor to which native PA binds, or to native LF.

The native target cell receptor for native PA is Anthrax Toxin Receptor (ATR)—see Bradley, K. A., et al (2001). Thus, in the context of the present invention, PA is substantially incapable of binding to ATR. Alternatively, in the context of the present invention, PA is incapable of binding to the native target cell receptor to which native PA binds if is substantially incapable of binding to monocyte or macrophage cells.

In order to confirm that any particular PA lacks a functional binding site for the native PA receptor on a target cell, a simple test may be performed as outlined in Example 10. Similarly, to confirm that any particular PA lacks a functional binding site for native LF, a simple test may be performed as outlined in Example 11.

In the context of the present invention, LF lacks a functional binding site if it is incapable of binding to a native PA. To confirm that any particular LF lacks a functional binding site for native PA, a simple test may be performed as outlined in Example 12.

The term “non-toxic” means that the components of the composition are substantially incapable of forming either active Lethal Toxin (LT) or active Oedema Toxin (ET). In this respect, an active toxin is one that is capable of binding to its native target cell, effecting translocation across the target cell membrane, and delivering enzymically active LF or EF into the cytosol thereof.

In use, the composition is substantially free of LT and ET activity.

A composition may be considered substantially non-toxic and substantially free of LT activity if the LT component of the composition possesses at most 20-%, preferably at most 10%, more preferably at most 5% of the activity of substantially pure, native LT (on a weight for weight basis). This may be determined by, for example, comparing respective LD₅₀ values, or by comparing respective cell lysis (eg. macrophage lysis) activities. The latter may be assessed based on the assay described in Example 9.

A composition may be considered substantially non-toxic and substantially free of ET activity if the ET component of the composition possesses at most 20%, preferably at most 10%, more preferably at most 5% of the activity of substantially pure, native ET (on a weight for weight basis). This may be determined by, for example, visually comparing respective tissue oedema-causing activities that are associated with ET.

Alternatively, the relative ET activities may be assessed by comparing respective intracellular adenyl cyclase activity. This may be assessed based on the assay described in Example 8.

According to a preferred embodiment of the present invention, the antigenic composition may include a third component, Oedema Factor (EF). The EF of the present invention preferably lacks a functional binding site, thereby preventing the EF from binding to native PA. In order to confirm that any particular EF lacks a functional binding site for native PA, a simple test may be performed as outlined in Example 13.

The PA, LF and EF components of the present invention that lack a functional binding site may be each prepared by modifying native PA, LF or EF (respectively) by conventional techniques. In this respect, the modification to provide a component lacking a functional binding site may be achieved at either the nucleic acid level or at the protein level. Structural modification of native PA, LF or EF is preferred.

For example, one or more of native PA, LF and EF may be subjected to conventional chemical or biological modification, eg. by toxoiding, so as to inactivate the native binding site in question. Also at the protein level, synthetic peptides may be employed that irreversibly bind to and thereby inactivate the binding site in question.

Alternatively, binding site inactivation may be achieved at the nucleic acid level by conventional non-specific mutagenesis or by conventional site-directed mutagenesis of nucleic acid encoding native PA, LF and EF. Suitable inactivation may be achieved by one or more deletion, insertion or substitution within the nucleic acid sequence encoding the binding site sequences, or within neighbouring sites that, in the resulting peptide, impose conformational changes on the binding site in question and thereby render said binding site dysfunctional.

The above protein level and nucleic acid level modifications are described in more detail later on in the present specification.

In a preferred embodiment, PA (ie. rather than LF or EF) lacks a functional binding domain, which substantially prevents PA from binding to either of LF or EF, or to its native target cell binding site. Optionally, PA may be further modified to reduce or substantially inactivate its native translocation function. Alternatively, in a separate aspect of the present invention, PA may be employed in a vaccine, wherein the PA has an inactive translocation domain but may possess native (ie. functional) binding domains.

An advantage associated with the inactivation of PA as the principal inactive component is that the antigenic composition of the present invention may then contain native (ie. active) LF and, if EF is present, native (ie. active) EF. This is possible because native PA is required for the formation of both active LT and active ET. The use of native toxin components may be preferred as such components possess the same epitopes associated with native toxin and therefore invoke a strong antigenic response.

LF, and EF (if present), each lacking a functional binding site may be employed in an antigenic composition of the present invention. Such binding site deficient LF and EF are not capable of binding to native PA via said binding site/s.

In one embodiment, the native enzyme activity function of LF and/or. EF may be substantially inactivated. Thus, in said embodiment the LF and/or EF of the present invention have at most 50%, preferably at most 25%, enzyme activity when compared (weight by weight) with native LF and EF, respectively.

However, in a preferred embodiment the native enzyme activity function of LF and/or EF is substantially retained. Thus, the LF of the present invention preferably retains at least 50%, more preferably at least 70% metalloprotease activity when compared (weight by weight) with native LF. Similarly, the EF of the present invention preferably retains at least 50%, more preferably at least 70% adenylyl cyclase activity when compared (weight by weight) with native EF.

The various components of the present invention are preferably prepared by recombinant means, thereby allowing the provision of a carefully defined composition. This is not possible with the current cell-free anthrax vaccine systems.

As detailed above, one or more of the PA, LF and EF molecules of the present invention lacks a functional binding site. This may be achieved by the introduction of a structural modification into or near to the binding site in question. For example, a molecule (eg. an alkyl group, or other steric hindrance molecule) may be incorporated into or near to the binding site to render the binding site dysfunctional. The same effect may be achieved by the introduction of a charged molecule that alters the charge environment within or near to the binding site. Alternatively, the whole binding site may be deleted, or specific amino acid residues may be deleted, substituted or inserted into or near to the binding site in question. However, it is preferred that the PA, LF and EF molecules of the present invention invoke an optimal immune response, and thus it is desirable that the process of binding site inactivation introduces minimal 3-D conformational changes outside of the binding site domain/s (ie. away from the binding site/s).

The binding site inactivation may be achieved at the DNA or protein level. In the latter case, suitable chemical or biological modifying agents may include:—alkylating agents; phosphorylating agents; general oxidising or reducing agents; aldehydes such as formaldehyde or glutaraldehyde; and peroxide generating agents such as hydrogen peroxide. Any of the modifying agents described in the Examples may be used to chemically or biologically modify one or more of PA, LF and EF.

When performing the binding site inactivation at the protein level, it is preferably to avoid undesirable 3-D conformational changes at positions unrelated to the binding site. Thus, in a preferred embodiment, the modifying agent, for example formaldehyde, is generally applied at a final concentration of approximately 0.1-5, preferably 0.2-1, typically 0.5% (v/v) to a composition of approximately 200 μg protein/ml. The modification process (also known as toxoiding) is then allowed to proceed at, for example, 37° C. for 5-20 hours, preferably 1-10 hours, more preferably 1-5 hours with occasional shaking.

Alternatively, binding site inactivation may be achieved at the nucleic acid level. For example, the individual components of the composition may be prepared recombinantly, during which process a modification may be introduced into one or more of the recombinant products. Such a modification substantially reduces the ability of a component of the present invention from forming active LT or ET.

Binding site inactivation of one component of the antigenic composition, particularly PA, is preferred. However, two or more components of the composition may be inactivated so as to lack a functional binding site.

Thus, in one embodiment, the composition comprises PA that is incapable of binding to LF or EF. This may be achieved by, for example, inactivating the furin cleavage site associated with native PA and thereby preventing exposure on PA of the LF or EF binding site in the first place, or by inactivating on PA the exposed LF or EF binding site.

Thus, in one embodiment the functional furin cleavage site (ie. amino acid residues 163-168) is inactivated. Furin is an enzyme that activates native PA (ie. the 83 kDa form) in vivo into the 63 kDa form by proteolytic cleavage, and thus exposes a specific binding site for which LF and EF compete in order to form LT and ET, respectively.

A single amino acid residue change (ie. deletion, insertion, or substitution) within or near to the furin cleavage site may reduce the effectiveness of furin cleavage and therefore substantially inactivate PA. In preferred embodiments, two or more amino acid residues are changed within the cleavage site, and in a particularly preferred embodiment the PA lacks the entire furin cleavage site (ie. all of residues 163-168 of native PA are missing). In another embodiment, one or more amino acid residues, or a short peptide sequence, may be inserted into the furin cleavage site. Any such short peptide sequence is preferably 1-20, more preferably 1-10, most preferably 1-5 amino residues in length.

In a related embodiment, PA is employed that lacks a functional binding site for its native target cell (eg. a modification is made within or near to amino acid residues 315-735, preferably within or near to residues 596-735 of Domain 4).

Thus, a change (ie. an amino acid deletion, substitution, or insertion) within or near to the PA binding site (eg. amino acid residues 315-735) may reduce the binding efficiency of PA for its native target cell receptor, and therefore substantially inactivate PA. As described above for the furin cleavage site inactivation of PA, two or more amino acid residues may be changed (ie. deleted, substituted, or inserted), including deletion of the entire PA binding site for its native target cell receptor, or a peptide sequence may be inserted into the binding site.

In a further embodiment, or in a separate aspect of the present invention, PA is employed that lacks a functional translocation domain.

In addition, or as an alternative to inactive PA, the composition of the present invention preferably comprises inactive LF.

In one embodiment, LF is employed that lacks a functional binding site for PA (eg. a modification is made within or near to the N-terminal Domain of LF, preferably within or near to amino acid residues 1-255).

In a further embodiment; LF is employed that lacks a functional endopeptidase activity or zinc-binding site (eg. a modification is made within or near to the C-terminal Domain of LF, preferably within or near to residues 686-692, which correspond to the native sequence “HEFGHAV”).

The level of LF endopeptidase activity may be assessed by the simple assay developed by the present Applicant (see Example 7). In this respect, preferred enzyme activity inactivation is achieved when the endopeptidase activity has been reduced to at most 40%, preferably 20%, more preferably 10% of the native LF activity.

The composition of the present invention may also comprise inactive EF.

In one embodiment, EF is employed that lacks a functional binding site for PA (eg. a modification is made within or near to the N-terminal Domain of EF, preferably within or near to amino acid residues 1-250).

In a further embodiment, or in addition to the binding site deficient embodiment, EF is employed that lacks adenylyl cy lase activity (eg. a modification is made within or near to the ATP-binding site occupied by residues 31.4-321, and/or within or near to the calmodium-binding site occupied by residues 613-767).

The level of EF adenyl cyclase activity may be assessed by an EF adenyl cyclase activity assay as described in Example 8. In this respect, preferred inactivation is achieved when the endopeptidase activity has been reduced to at most 40%, preferably 20%, more-preferably 10% of the native EF activity.

According to a separate aspect of the present invention, inactive EF may be employed as a principal vaccine component, optionally with PA and/or LF. The EF is inactive in terms of adenylyl cyclase activity and/or has an inactive binding site for PA. The PA and/or LF components may lack a functional binding site as described in detail above, and may be accompanied by other antigens such as Sap and/or EA1.

A further means for rendering the PA, LF and EF components of the present invention dysfunctional in terms of binding site function is to include an inhibitor that inactivates the binding site/s on one or more of PA, LF and EF.

In one embodiment, the inhibitor mimics the binding site on PA for its native target cell, or for LF or EF. Alternatively, the inhibitor may bind to the furin cleavage site on PA. Thus, following binding of the inhibitor to PA, the ability of PA to bind is native target cell receptor, or to LF or EF, or to translocate LF or EF is substantially reduced or inhibited, thereby rendering PA inactive. The inhibitor preferably binds irreversibly to PA. In one embodiment, the inhibitor is a short peptide possessing the motif “YWWL”. Preferred embodiments include HTSTYWWLDGAP” and “HQLPQYWWLSPG”.

In another embodiment, the inhibitor mimics the binding site on either LF or EF for PA. Thus, following binding of the inhibitor to LF or EF, the ability of LF or EF to bind PA, is substantially reduced or inhibited, thereby rendering LF or EF inactive. The inhibitor preferably binds irreversibly to LF or EF. In one embodiment, the inhibitor binds an active site on LF or EF, or removes/prevents the binding of cofactors to LF or EF (eg. zinc, ATP, calcium, and/or calmodium).

The composition of the present invention may contain additional antigenic components, preferably one or more S-layer protein.

The best characterised of the somatic antigens of B. anthracis are the S-layer proteins, Sap (eg. Sap 1) and EA1 [see Farchaus et al., (1995) J. Bacteriology, 177, pp. 2481-2489; and Mesnage et al. (1997) Molec. Microbiol. 23, pp. 1147-1155].

There is currently no clear biological function associated with the S-layer proteins of B. anthracis. At the protein sequence level their N-terminal regions display up to 66% identity. In contrast, their C-terminal regions have little identity or similarity.

Whilst both Sap and EA1 are cell-associated, EA1 constitutes the major cell-associated antigen.

The Sap protein is produced at high levels by B. anthracis “Sterne” derivatives during growth in vitro. Although antisera from animals presented with B. anthracis. “Sterne” strain derivatives apparently recognise EA1, cell extracts containing the S-layer proteins have been reported not to provide protection against challenge with virulent B. anthracis strains.

The origin and source of antigens in the composition of the present invention is preferably the natural host (ie. B. anthracis). This is because production of antigens in a different host may lead to variation in the protein conformation resulting from changes in translation fidelity and in accurate post-translational modification. Such changes could lead to an alteration of the antigenicity or immunogenicity of these antigens.

However, in view of strain-by-strain variance of PA, LF and EF, preferred embodiments of the present invention employ multivalent PAs, LFs and optionally EFs having varied conformations or epitopes. In addition to the full-length conserved antigens, the presence of immunogenic breakdown products may be preferred.

According to a second aspect of the present invention there is provided a composition comprising Protective Antigen (PA) and Lethal Factor (LF), wherein PA and LF are each present at a concentration of 1-60 μg/ml, preferably 2-40 μg/ml, more preferably 2-20 μg/ml.

In a preferred embodiment, the minimum concentration of each of PA and LF is 2, preferably 5, more preferably 10 μg/ml.

In one embodiment, the composition comprises PA and LF in weight ratios of 1:3 to 3:1, preferably 1:2 to 2:1, more preferably 1:1.5 to 1.5:1.

The composition components may be derived directly from a culture of native B. anthracis or by mixing appropriate quantities of recombinant antigens.

The above components (in the indicated ratios and/or concentrations) together with Oedema Factor (EF), if present, may each lack a functional binding site as described above for the first aspect of the present invention.

According to a third aspect of the present invention, there is provided a method for preparing a composition comprising:—
culturing B. anthracis bacteria in a medium comprising at least 10 mM glucose;
harvesting the bacteria at a time point when the glucose concentration has been reduced to a concentration of up to 1 mM, preferably up to 0.5 mM, more preferably up to 0.1 mM;
adding a compound that precipitates soluble or suspended proteins, thereby forming a culture precipitate; and
recovering the precipitate that contains Protective Antigen (PA), Lethal Factor (LF), and optionally Oedema Factor (EF).

The compound that precipitates soluble or suspended proteins is preferably potassium aluminium sulphate.

The above components together with Oedema Factor (EF), if present, may each lack a functional binding site as described above for the first aspect of the present invention.

The method of the third aspect may be employed to prepare a composition having the component concentrations as defined for the second aspect of the present invention.

A fourth aspect of the present invention provides a recombinant method for preparing an antigenic composition, said method comprising:—
expressing in a B. anthracis host cell a nucleic acid construct encoding Protective Antigen (PA), and recovering the expressed PA;
expressing in a B. anthracis host cell a nucleic acid construct encoding Lethal Factor (LF), and recovering the expressed LF;
optionally expressing in a B. anthracis host cell a nucleic acid construct encoding Oedema Factor (EF), and recovering the expressed EF; and
combining the PA, LF and optionally EF;
wherein the PA and/or LF lacks a functional binding site.

Sap 1 and/or EA 1 may be prepared similarly, and added to the antigenic composition.

Preferably PA lacks a functional binding site. Alternatively, PA together with LF (and optionally EF) each lacks a functional binding site.

In a preferred embodiment, the components are present in the concentrations and optionally in the ratios described above for the second aspect.

In view of the recombinant nature, and binding site modifications, the conformation of the components of the antigenic composition may be slightly different from the native PAs, LFs and EFs of infecting strains. This may provide improved antigenicity against a range of infecting strains.

According to a fifth aspect of the invention, there is provided an antibody that binds to at least one of PA, LF, and EF, and when so bound thereto, the PA, LF or EF (respectively) lacks a functional binding site. The antibody aspect of the present invention is preferably employed post-infection.

The antibody preferably has specificity for the binding site in question.

In one embodiment, a composition is provided that comprises two or more of said antibodies, which antibodies bind to different molecules selected from PA, LF or EF. Antibodies that bind to Sap 1 or EA1 may be also included.

If polyclonal antibodies are desired, a selected mammal (eg. mouse, rabbit, goat, horse, etc.) is immunized with an immunogenic polypeptide. Serum from the immunized animal is collected and treated according to known procedures. If serum containing polyclonal antibodies to a desired epitope contains antibodies to other antigens, the polyclonal antibodies may be purified by immunoaffinity chromatography.

Alternatively, general methodology for making monoclonal antibodies by hybridomas involving, for example, preparation of immortal antibody-producing cell lines by cell fusion, or other techniques such as direct transformation of B lymphocytes with oncogenic DNA, or transfection with Epstein-Barr virus may be employed.

The antibody employed in this aspect of the invention may belong to any antibody isotype family, or may be a derivative or mimic thereof. Reference to antibody throughout this specification embraces recombinantly produced antibody, and any part of an antibody that is capable of binding to the anthrax antigen in question.

In one embodiment the antibody belongs to the IgG, IgM or IgA isotype families.

In another embodiment, the antibody belongs to the IgA isotype family. Reference to the IgA isotype throughout this specification includes the secretory form of this antibody (ie. sIgA). The secretory component (SC) of sigA may be added in vitro or in vivo. In the latter case, the use of a human's natural SC labelling machinery may be employed.

The antibody of the present invention may be polyclonal, but is preferably monoclonal.

According to a sixth aspect of the invention, there is provided a DNA plasmid that encodes PA or LF, which PA or LF lacks a functional binding site thereby preventing said PA and LF from binding together via said binding site or thereby preventing said PA from binding to a native PA cell receptor via said binding site, and wherein said plasmid includes a eukaryotic promoter that is operably linked to and drives expression of said PA or LF, respectively. Thus, the DNA plasmid may be employed as a DNA vaccine, and may include a polyadenylation signal.

According to an alternative aspect of the present invention, there is ‘provided a DNA’ plasmid that encodes EF, which EF lacks a functional binding site thereby preventing said EF from binding to PA or which EF substantially lacks adenylyl cyclase activity, and wherein said plasmid includes a eukaryotic promoter that is operably linked to and drives expression of said EF.

In one embodiment of the DNA plasmid aspects of the present invention, there is provided a plasmid (or plasmids) that encodes and permits expression of two or more of said aforementioned PA, LF or EF, and optionally Sap 1 and/or EA1.

The DNA plasmids of the present invention are preferably administered as a vaccine.

In a related aspect, the present invention provides an RNA molecule that encodes at least one of said aforementioned PA, LF or EF, Sap 1.

In one embodiment of the RNA molecule aspect of the present invention, there is provided two or more of said RNA molecules that encode and permit expression of two or more of said aforementioned PA, LF or EF, and optionally Sap 1 and/or EA1.

The RNA molecule/s may be introduced as a vaccine directly into an animal, preferably a human, or may be incorporated into an RNA vector prior to administration.

A seventh aspect of the invention provides use of the antigenic composition, the antibodies, and/or the DNA- or RNA-containing compositions defined herein, in the manufacture of a medicament for substantially preventing anthrax poisoning.

The various antigenic compositions described in the present application are intended for use as a vaccine.

The vaccine components (eg. PA, LF, and EF) may be administered prior to, or simultaneously with, or subsequent to one another.

The vaccine may be administered by conventional routes, eg. intravenous, subcutaneous, intraperitoneal, and mucosal routes.

Typically, such vaccines are prepared as injectables, either as liquid solutions or suspensions; solid forms suitable for solution in, or suspension in, liquid prior to injection may also be prepared. The preparation may also be emulsified, or the peptide encapsulated in liposomes or microcapsules.

The active immunogenic ingredients are often mixed with excipients which are pharmaceutically acceptable and compatible with the active ingredient. Suitable excipients are, for example, water, saline, dextrose, glycerol, ethanol, or the like and combinations thereof. In addition, if desired, the vaccine may contain minor amounts of auxiliary substances such as wetting or emulsifying agents, pH buffering agents, and/or adjuvants which enhance the effectiveness of the vaccine. Examples of adjuvants which may be effective include but are not limited to: aluminum hydroxide, N-acetyl-muramyl-L-threonyl-D-isoglutamine (thr-MDP), N-acetyl-nor-murarnyl-L-alanyl-D-isoglutamine (CGP 11637, referred to as nor-MDP), N-acetylmuramyl-L-alanyl-D-isoglutaminyl-L-alanine-2-(1 w-2′-dipalmitoyl-sn-glycero-3-hydroxyphosphoryloxy)-ethylamine (CGP 19835A, referred to as MTP-PE), and RIBI, which contains three components extracted from bacteria, monophosphoryl lipid A, trehalose dimycolate and cell wall skeleton (MPL+TDM+CWS) in a 2% squalene/Tween 80 emulsion.

The vaccines are conventionally administered parenterally, by injection, for example, either subcutaneously or intramuscularly.

The vaccines are administered in a manner compatible with the dosage formulation, and in such amount as will be prophylactically and/or therapeutically effective. The quantity to be administered, which is generally in the range of 5 micrograms to 250 micrograms of antigen per dose, depends on the subject to be treated, capacity of the subject's immune system to synthesize antibodies, and the degree of protection desired. Precise amounts of active ingredient required to be administered may depend on the judgment of the practitioner and may be particular to each subject.

The vaccine may be given in a single dose schedule, or optionally in a multiple dose schedule. A multiple dose schedule is one in which a primary course of vaccination may be with 1-6 separate doses, followed by other doses given at subsequent time intervals required to maintain and or reinforce the immune response, for example, at 1-4 months for a second dose, and if needed, a subsequent dose(s) after several months. The dosage regimen will also, at least in part, be determined by the need of the individual and be dependent upon the judgment of the practitioner.

In addition, the vaccine containing the immunogenic antigen(s) may be administered in conjunction with other immunoregulatory agents, for example, immunoglobulins, as well as antibiotics.

Additional formulations which are suitable for other modes of administration include microcapsules, suppositories and, in some cases, oral formulations or formulations suitable for distribution as aerosols. For suppositories, traditional binders and carriers may include, for example, polyalkylene glycols or triglycerides; such suppositories may be formed from mixtures containing the 20‘active ingredient ’ in the range of 0.5% to 10%, preferably 1% 2%.

Oral formulations include such normally employed excipients as, for example, pharmaceutical grades of mannitol, lactose, starch, magnesium stearate, sodium saccharine, cellulose, magnesium carbonate, and the like. These compositions take the form of solutions, suspensions, tablets, pills, capsules, sustained release formulations or powders and contain 10%-95% of active ingredient, preferably 25%-70%.

In one embodiment the medicament may be administered intranasally (i.n.). An intranasal composition may be administered in droplet form having approximate diameters in the range of 100-5000 μm, which in terms of volume would have droplet sizes in the approximate range of 0.01-100 μl.

Intranasal administration may be achieved by way of applying nasal droplets or via a nasal spray. In the case of nasal droplets, the droplets may typically have a diameter of approximately 1000-3000 μm and/or a volume of 1-25 μl, whereas in the case of a nasal spray, the droplets may typically have a diameter of approximately 100-1000 μm and/or a volume of 0.001-1 μl.

It is possible that, following i.n. delivery of antibodies, their passage to the lungs may facilitated by a reverse flow of mucosal secretions.

In a different embodiment, the medicament may be delivered in an aerosol formulation. The aerosol formulation may take the form of a powder, suspension or solution.

The size of aerosol particles is one factor relevant to the delivery capability of an aerosol. Thus, smaller particles may travel further down the respiratory airway towards the alveoli than would larger particles. In one embodiment, the aerosol particles have a diameter distribution to facilitate delivery along the entire length of the bronchi, bronchioles, and alveoli. Alternatively, the particle size distribution may be selected to target a particular section of the respiratory airway, for example the alveoli.

The aerosol particles may be delivered by way of a nebulizer or nasal spray.

In the case of aerosol delivery of the medicament, the particles may have diameters in the approximate range of 0.1-50 μm, preferably 1-5 μm.

The aerosol formulation of the medicament of the present invention may optionally contain a propellant and/or “surfactant.

By controlling the size of the droplets which are to be administered to a patient to within the defined range of the present invention, it is possible to avoid/minimise inadvertent antigen delivery to the alveoli and thus avoid alveoli-associated pathological problems such as inflammation and fibrotic scarring of the lungs.

I.n. vaccination engages' both T and B cell mediated effector mechanisms in nasal and bronchus associated mucosal tissues, which differ from other mucosae-associated lymphoid tissues.

Intranasal delivery of antigens allows targeting of the antigens to submucosal B cells of the respiratory system. These B cells are the major local IgA-producing cells in mammals and intranasal delivery facilitates a rapid increase in IgA production by these cells against the anthrax antigens.

In one embodiment administration of the medicament comprising an anthrax antigen stimulates IgA antibody production, and the IgA antibody binds to the anthrax antigen. In another embodiment, a mucosal and/or Th2 immune response is stimulated.

Reference throughout the present application to the components PA, LF, EF, and the S-layer proteins embraces fragments, variants and derivatives thereof.

The term “fragment” means a peptide having at least five, preferably at least ten, more preferably at least twenty, and most preferably at least thirty-five amino acid residues of the component in question. The fragment preferably includes at least one epitope of the corresponding native component. The fragment may result from enzymic break-down of the corresponding native component.

The term “variant” means a peptide or peptide fragment” having at least seventy, preferably at least eighty, more preferably at least ninety percent amino acid sequence homology with the component in question. An example of a “variant” is a peptide or peptide fragment which contains one or more analogs of an amino acid (eg. an unnatural amino acid), or a substituted linkage. The terms “homology” and “identity” are considered synonymous in this specification.

For sequence comparison, typically one sequence acts as a reference sequence, to which test sequences may be then compared. When using a sequence comparison algorithm, test and reference sequences are input into a computer, subsequent coordinates are designated, if necessary, and sequence algorithm program parameters are designated. The sequence comparison algorithm then calculates the percentage sequence identity for the test sequence(s) relative to the reference sequence, based on the designated program parameters.

Optimal alignment of sequences for comparison may be conducted, for example, by the local homology alignment algorithm of Smith and Waterman [Adv. Appl. Math. 2: 484 (1981)], by the algorithm of Needleman & Wunsch [J. Mol. Biol. 48: 443 (1970)] by the search for similarity method of Pearson & Lipman [Proc. Nat'l. Acad. Sci. USA 85: 2444 (1988)], by computer implementations of these algorithms (GAP, BESTFIT, FASTA, and TFASTA —Sequence Analysis Software Package of the Genetics Computer Group, University of Wisconsin Biotechnology Center, 1710 University Avenue, Madison, Wis. 53705), or by visual inspection [see Current Protocols in Molecular Biology, F. M. Ausbel et al, eds, Current Protocols, a joint venture between Greene Publishing Associates, In. And John Wiley & Sons, Inc. (1995 Supplement) Ausbubel].

Examples of algorithms suitable for determining percent sequence similarity are the BLAST and BLAST 2.0 algorithms [see Altschul (1990) J. Mol. Biol. 215: pp. 403-410; and “http://www.ncbi.nim.nih.gov/” of the National Center for Biotechnology Information].

In a preferred homology comparison, the identity exists over a region of the sequences that is at least 10 amino acids, preferably at least 20 amino acids, more preferably at least 35 amino acids in length.

The term “derivative” means a molecule comprising the component (or fragment, or variant thereof) in question. Thus, a derivative may include the component in question, and a further sequence (eg. a peptide) that introduces one or more additional epitopes. The further sequence should preferably not interfere with the basic folding and thus conformational structure of the peptide in question.

Examples of a “derivative” are a fusion protein, and a conjugate. Thus, two or more components (or fragments, or variants) may be joined together to form a derivative. Alternatively, a component (or fragment, or variant) may be joined to an unrelated molecule (eg. a second, unrelated peptide). Derivatives may be chemically synthesized, but will be typically prepared by recombinant nucleic acid methods. Additional non-peptide molecules such as lipid, and/or polysaccharide, and/or polyketide components may be included in a derivative.

All of the molecules “fragment”, “variant” and “derivative” have a common antigenic cross-reactivity and/or substantially the same in vivo biological activity as the component from which they are derived. For example, an antibody capable of binding to a fragment, variant or derivative would be also capable of binding to the component in question.

It is a preferred feature that the fragment, variant and derivative each possess the active site (eg. binding site, or enzyme function active site) of the component in question. Thus, in the case of LF, such a fragment, variant or derivative thereof possesses the endopeptidase active site and/or zinc-binding site of LF. Similarly, in the case of EF, such a fragment, variant or derivative thereof possesses the adenylyl cyclase active site of EF. Preferably, said fragments, variants or derivatives of LF possess at least 30% native metalloprotease activity, and said fragments, variants or derivatives of EF possess at least 30% native adenylyl cyclase activity.

Referring to the Figures of the present application:—

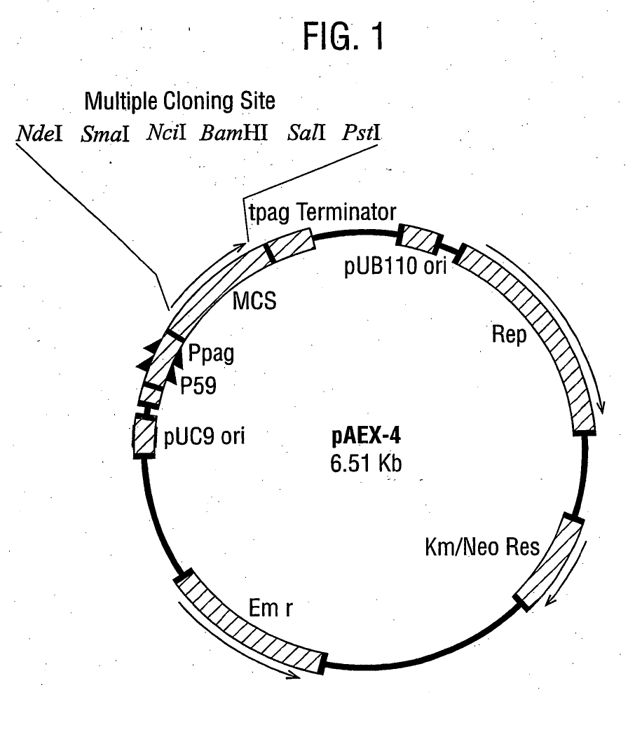

FIG. 1 illustrates a plasmid map of pAEX-4, which is a shuttle vector capable of replication in both E. coli and a variety of Gram positive bacilli such as B. anthracis. The construct is approximately 6.5 Kb in size and comprises the replication functions of pUC9 (for replication in E. coli) and pUB110 (for replication in B. anthracis). Additionally, selectable markers encoding resistance to neomycin/kanamycin (in Bacillus) and erythormycin (in E. coli) are present. Expression of target genes is driven by a tandem combination of the lactococcal P59 and protective antigen Pag A (Ppag) promoters. The transcriptional terminator t_{pag} is derived from the B. anthracis pagA gene. Translation is initiated from the staphylococcal protein A ribosome binding site positioned upstream from the protein A signal sequence. The multiple cloning site (MCS) has an Nde1 site for cloning of the toxin component genes. Recombinant proteins are produced from this vector without a fusion partner;

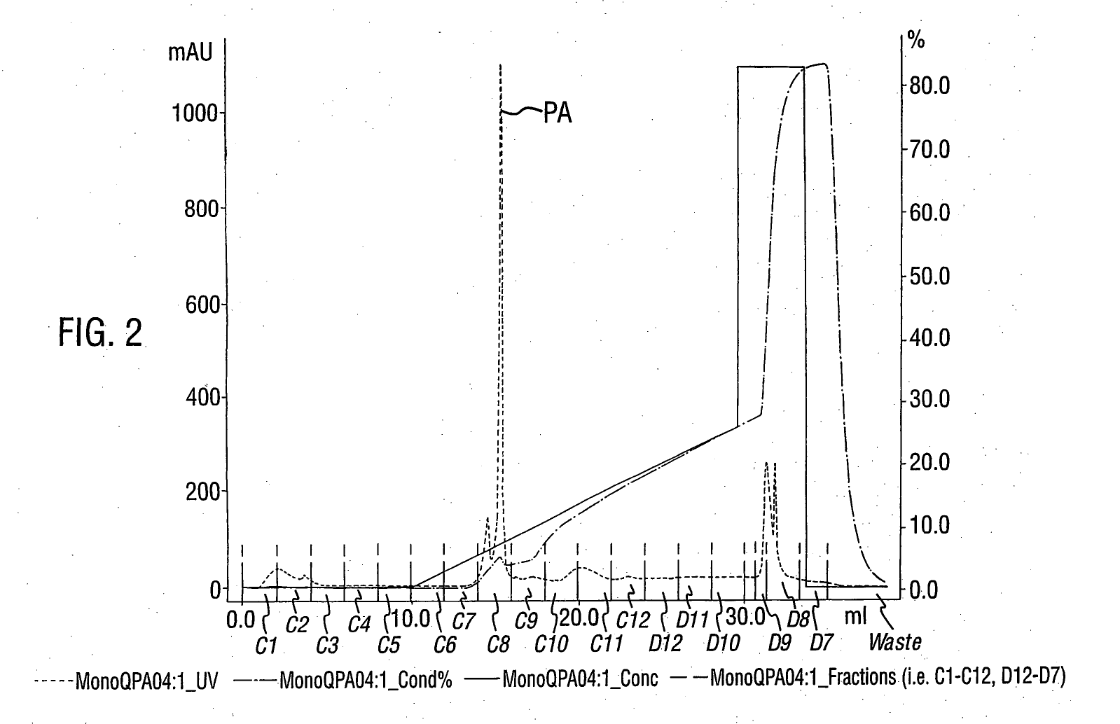

FIG. 2 illustrates an elution profile of recombinant PA by Ion Exchange Chromatography using an XK26/20 Source 30Q column; and

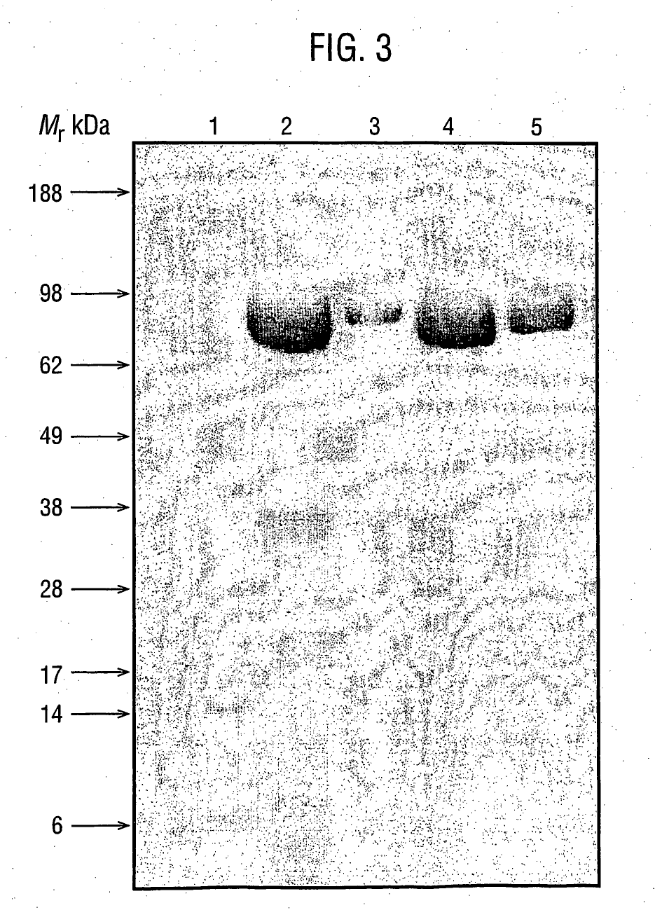

FIG. 3 illustrates an SDS-PAGE analysis of the purification steps employed in preparing recombinant LF. Lane 1 shows Invitrogen Seeblue (registered trademark) plus markers; lane 2 shows crude culture supernatant; and lanes 3-5 show factions across LF peak (Source 30Q Anion-exchange column). Peak fraction in track 4 is >96% pure as determined by scanning densitometry).

The invention is now described by reference to the following Examples.

### EXAMPLE 1

### Production of Recombinant Antigens

A non-toxigenic strain of Bacillus anthracis (strain UM23C1-1) has been successfully used as a host for the recombinant expression of anthrax toxin genes. Use of the native host affords clear advantages of gene expression in a natural genetic background. This host was, therefore, used for expression of the toxin component reagents required for this study.

Two expression vectors (pAEX-4 & pAEX-AV4) have been constructed and differ from each other in the use of different promoter combinations and in the provision of a purification tag (pAEX-AV4). Both vectors are Gram-negative/Gram-positive shuttle vectors capable of replication in both. E. coli, for the purpose of routine cloning operations, and in B. anthracis UM23C1-1 for expression of recombinant proteins. As a representative of the two vectors, pAEX-4 is illustrated diagrammatically in FIG. 1.

### EXAMPLE 2

### Development of B. anthracis Expression Systems: Plasmid pAEX-4

The expression vector pAEX-4 provides a source of recombinant lethal factor (LF), oedema factor (EF) and protective antigen (PA) without a fusion partner for purification.

In this system the lactococcal P59 promoter and the B. anthracis Ppag (protective antigen) promoters in tandem, drive expression. Replication in E. coli is initiated from the pUC9 origin of replication whilst in Gram-positive hosts it is initiated from the well-characterised pUB110 origin.

Sub-cloning anthrax toxin genes:—

The toxin genes cya, lef & pag encoding EF, LF and PA respectively were sub-cloned from appropriate clones obtained from the CAMR nucleic acid collection. The genes were removed as NdeI-SalI fragments by restriction endonuclease digestion. The 5′ and 3′ termini of all genes were generated by Polymerase Chain Reaction (PCR) using oligonucleotides designed to incorporate the required restriction endonuclease recognition sites. All sub-cloning work was performed using commercially available E. coli K12 derivative hosts. Prior to transformation into B. anthracis for expression analysis, DNA constructs were passaged through the dam⁻ dcm⁻ E. coli host SCS110 (Stratagene, Europe).

### EXAMPLE 3

### Production of Antigen Seed Stocks

B. anthracis UM23C1-1 harbouring either PAEX-4pag, pAEX-4lef or pAEX-4cya are grown in modified PYS5 medium containing 10 μg/ml neomycin for 16 hours at 37° C. with moderate aeration (200 rpm, 200 ml volume in a 2000 ml flask). After overnight growth, 0.5 ml aliquots of the appropriate culture are mixed with 0.5 ml of 40% (v/v) sterile glycerol and stored at −70° C. until required.

Viability is assessed by inoculation and growth of cultures from the seed stocks and comparing expression levels of protective antigen (PA), lethal factor (LF) antigen or oedema factor (EF) antigen with cultures inoculated directly from colonies of B. anthracis UM23C1-1 freshly transformed with the appropriate expression construct.

### EXAMPLE 4

### Batch Culture Growth of B. anthracis UM23C1-1

B. anthracis UM23C1-1 clones harbouring the appropriate expression construct (see FIG. 1) are grown in modified-PYS5 medium containing 10 μg/ml neomycin for 16 hours at 37° C. with moderate aeration (200 rpm, 500 ml volume in a 2000 ml flask). After overnight growth the 500 ml cultures of B. anthracis are harvested by centrifugation (10,000×g, 10 min, 4° C. Sorvall RC5B34), the supernatants are chilled on ice and filter sterilised under vacuum (Millipore, 0.22 μm PVDF membrane). All supernatants are stored at −20° C. pending analysis and antigen purification.

### EXAMPLE 5

### Antigen Purification

PA is expressed in B. anthracis UM23C1-1 harbouring the plasmid pAEX-4pag. Culture supernatants were clarified by centrifugation and sterile filtered using a 0.22 μM nitrocellulose filter (Millipore). Solid ammonium sulphate was slowly added with stirring to culture supernatants, to a final concentration of 60% saturation. Precipitated proteins are recovered by centrifugation. (10,000×g, 4° C., 10 min).

Pellets are resuspended in 20 mM piperazine, pH 9.7, containing 1 mM EDTA and dialysed overnight against an excess of the same buffer. The dialysate is applied to a Source 30Q anion-exchange column (AP Biotech) equilibrated in the same buffer. Proteins are eluted using a NaCl gradient developed in 20 mM piperazine, pH 9.7, containing 1 mM EDTA as above (see FIG. 2). Fractions containing PA are identified by SDS-PAGE (see FIG. 3) and western blotting using rabbit anti-PA antiserum.

LF and EF are expressed and purified as described above for PA with slight modifications.

The typical yields of these proteins using the above growth conditions are 80 mg/l, 35 mg/l and 5 mg/l for PA, LF and EF respectively.

### EXAMPLE 6

### Formulation of Anthrax Vaccine Composition

The vaccine may be formulated by either:—
1. combining appropriate quantities of purified recombinant antigens produced in their native host, B. anthracis; or
2. directly from native B. anthracis cultures.

Option 1 is now described in more detail, and employs appropriate quantities of purified recombinant antigens.

The principal components of the vaccine are the two anthrax toxin components, protective antigen (PA) and lethal factor (LF).

The principal components may be combined with other proteins such as EF, Sap, EA-1 etc. Adjuvants such as Alhydrogel may be added to the combined protein mixture or to the individual proteins prior to combining. These components (and other proteins) are combined together at a preferred concentration such as 1 to 20 μg/human dose.

The combined proteins are preferably formulated in a way that ensures the safety (ie. non-toxicity) of the vaccine.

In the following illustration, this is achieved by inactivation of PA by replacing the rPA with rΔPA83.
PA is an 83 kDa protein (PA83) which, after binding to its target cell surface receptor, is proteolytically activated to a 63 kDa species (PA63). It is PA63 that binds with high affinity to LF or EF and forms the lethal toxin or oedema toxin respectively.
rΔPA83 is a form of PA which lacks amino acid residues 163-168 (proteolytic cleavage site) and so is not activatable to the PA63 form. Moreover rΔPA83 is unable to bind to LF or EF in the proposed vaccine, and therefore the lethal and oedema toxins can not be formed.

In the following illustration, this is achieved by toxoiding the individual components or the final mixture using formaldehyde in order to inactivate their biological activities.
Dilute antigen to 200 μg/ml in 0.05 M phosphate, 0.5 M NaCl, pH 7.2. Whilst stirring in a beaker, slowly add sufficient 40% formaldehyde to a final concentration of 0.5%. Transfer to a screw cap bottle, and incubate at 37° C. for 7-14 days with occasional shaking. Dialyse material 3-5 times against 10-20 volumes of phosphate buffered saline (PBS) at 4° C. for 12 hours. Residual levels of formaldehyde (0.01%) may be added.

Option 2 is now described in more detail, and employs native B. anthracis cultures.

The vaccine is formulated directly from toxigenic, non-capsulating B. anthracis 34F2 “Sterne” strain cultures.

Cultures may be grown in either a partially defined or a complex medium that supports the growth of B. anthracis and the production of the preferred vaccine components. Growth is performed under optimum conditions and culture harvest markers will be monitored. These harvest markers will be identified to provide the production of appropriate quantities of the preferred vaccine components. The markers can be, for example, when the culture pH value falls below pH 7.4 or glucose concentration falls below 1 mM.

At the end of the growth period (approximately 24-28 hours) the cultures are harvested and the pooled supernatants filter-sterilised through a 0.2 micron filter. The supernatant can be precipitated using potassium aluminium sulphate and the pH adjusted to the required value.

As with Option 1, the composition may be rendered non-toxic by toxoiding.

### EXAMPLE 7

### LF Endopeptidase Assay

The assay employs a synthetic peptide substrate representing the N-terminal 0.60 residues of human MEK-1. Samples containing native or recombinant LT (ie. LF) are diluted in assay buffer (25 mM potassium phosphate buffer, pH 7.0, containing 0.05 mM ZnSO₄ and 0.05 mM CaCl₂) and incubated with the substrate.

Rabbit antiserum produced against cleaved peptide (representing the N-terminal amino acid residues generated following cleavage of MEK-1 by LF) is then used to measure the enzymic/biological activity of LF.

### EXAMPLE 8

### EF and ET Adenylyl Cyclase Assays

Adenylate cyclase activity (inherent to EF, and ET) may be determined by the measurement of either extracellular and intracellular cAMP production resulting from oedema factor (EF) activity or from oedema toxin (ET; PA+EF) activity, respectively.

Adenylate cyclase activity can be determined as follows. Briefly, reaction mixtures containing 20 μl of 5× assay buffer (100 mM-HEPES pH 7.5, 25 mM Mn Cl₂, 2.5 mM CaCl₂, 2.5 mM EDTA, 2.5 mM dithiothreitol, 0.5 mg/ml bovine serum albumin), 5 μl of bovine calmodulin (100 μg/ml), 10 μl of 20 mM ATP, 10 μl of EF (dilutions of 1 ng/μl), and water to 100 μl are set up in triplicate and incubated for 60 min at 30° C.

Commercial kit assays eg the BIOTRAK cAMP enzyme immunoassay (EIA) system kit from Amersham-Pharmacia is used to measure the cAMP concentration.

### EXAMPLE 9

### Macrophage Lysis Assays for LF and PA

The monocyte/macrophage cells RAW 264.7 were obtained from ECACC (CAMR) and maintained in Dulbecco's modified Eagle's medium (DMEM) with 3% (v/v) L-glutamine, 10% (v/v) foetal calf serum and Penicillin/Streptomycin antibiotics solution at 0.5 IU/ml and 0.5 μg/ml respectively. The cells were routinely grown in 75 cm² flasks at 37° C. in a humidified 5% (v/v) carbon dioxide (CO₂) atmosphere.

The macrophage lysis assay has been established as described below. Briefly, RAW 264.7 monocyte/macrophages were harvested by scraping growing cultures into pre-warmed (37° C.) DMEM buffered with 10 mM HEPES, pH 7.4 (DMEM/HEPES) and adjusting the cell density to 5×10⁵ cells/ml. The cell suspension was plated at 200 μl/well (1×10⁵ cells/well) in 96-well culture plates and cells were allowed to settle and attach for 16 hours at 37° C., 5% CO₂.

To begin the assay for quantification of LF, medium and detached cells were removed by gentle aspiration and replaced (100 μl/well) with warm DMEM/HEPES containing 0.1 μg/ml of PA. LF was then added at different concentrations in DMEM/HEPES containing 0.1 μg/ml PA. All experiments were done in triplicate (unless otherwise indicated) over a 100-fold concentration range. Cell viability was determined after a 3 hour incubation period with toxin using the 3-[4,5-dimethylthiazol-2-yl]-2,5-diphenyl-tetrazolium bromide (MTT) tetrazolium dye assay.

MTT (Sigma, UK) was dissolved in DMEM/HEPES at 1.5 mg/ml and warmed to 37° C. before addition to cell cultures (100 μl/well) to effect a final concentration of 0.5 mg/ml. Incubation was continued at 37° C., 5% CO₂ for 60 min to allow uptake and oxidation of the dye by viable cells. Medium was aspirated and replaced by 100 μl/well of 0.5% sodium dodecyl sulphate (w/v), and 25 mM HCl in 90% isopropyl alcohol and the plates shaken to disrupt the cells and dissolve the MTT (10-30 mins).

After visual inspection to ensure dissolution of MTT crystals, MTT absorption at 570 nm was determined using a Dynatech MR7000 plate reader.

The macrophage lysis assay described above may be used for detection/quantification of PA but using a fixed concentration of LF (0.1 μg/ml).

### EXAMPLE 10

### To Show that Modified PA does not Bind to its Cellular Target

Anthrax toxin receptors (the cellular target to which PA binds) are ubiquitous and expressed at moderately high levels on cell surfaces, even on cell lines that are not sensitive to the effects of lethal toxin (Bradley et al, 2001).

Receptor binding assays using radiolabelled PA are employed to confirm that any particular modified PA according to the present invention does not bind to these receptors in a defined cell line, eg. mouse macrophage J774A.1 (lethal toxin sensitive) or macrophage A/J (resistant) (Freidlander et al, 1993).

In more detail, cells are plated into 96 well tissue culture plates at approximately 3×10⁵ cells/ml and exposed to radio iodinated PA 83 (control) or modified PA at 4° C. for one hour to allow binding to occur. The low incubation temperature prevents internalisation of bound PA. Cells are then washed 3 times with cold PBS to remove unbound labelled PA, the cells solubilised and radioactivity in the resulting samples quantified using a gamma counter.

### EXAMPLE 11

### To Show that Modified PA does not Bind to LF or to EF

Modified PA may be susceptible to protease cleavage, in the same way as PA83 can be cleaved, either naturally at the cell surface by furin, or artificially by trypsin or chymotrypsin, to form a derivative analogous to PA63 but which derivative does not bind to LF or EF. Alternatively, the modified PA is simply not susceptible to protease cleavage.

This can be tested if 1 to 5 mg of the modified PA is cleaved by incubation with, for example, trypsin (20 μg) for 30 min at 37° C. in 1.5 ml of a suitable buffer (eg. 20 mM ethanolamine at pH 9). The reaction is then stopped by adding trypsin inhibitor (40 μg in 10 μl of buffer).

The fragments are then be purified by conventional low pressure liquid chromatography (eg. gel filtration or anion exchange chromatography) and the fragment corresponding to PA63 coated on to microtitre plates as described in Example 12.

After blocking the plates, dilutions of LF or EF are allowed to bind to the modified PA and bound antigens quantified as described in Example 12.

### EXAMPLE 12

### To Show that Modified LF does not Bind to PA

This is confirmed by coating PA63 on to microtitre plates at a concentration of 1 μg/ml. Antigen (PA63) is coated overnight at 2-8° C. at 1 μg/ml in carbonate/bicarbonate buffer pH 9.6. The coated plates are washed and blocked by the addition of diluent (phosphate buffered saline containing 0.1% Tween and 5% Foetal Calf Serum) to all wells.

Dilutions of LF (control) or modified LF are added in blocking buffer. Binding is allowed to proceed for 60 minutes at 37° C. Plates are washed 4 times in PBS-T and bound LF detected using an HRP-conjugated anti-LF antibody. Excess antibody is removed by washing as above and the plates developed by the addition of 100 μl substrate to each well.

The colour development reaction is stopped by the addition of 50 μl NaOH (3M) and the absorbance read at 405 and 690 nm.

### EXAMPLE 13

### To Show that Modified EF does not Bind to PA

This is confirmed by coating PA63 on to microtitration plates at a concentration of 1 μg/ml. Antigen (PA63) is coated overnight at 2-8° C. at 1 μg/ml in carbonate/bicarbonate buffer pH 9.6. The coated plates are washed and blocked by the addition of 50 μl diluent (phosphate buffered saline containing 0.1% Tween, registered trademark, and 5% foetal calf serum) to all wells.

Dilutions of EF (control) or modified EF are added in blocking buffer. Binding is allowed to proceed for 60 minutes at 37° C. Plates are washed 4 times in PBS-T and bound EF detected using an HRP-conjugated anti-EF antibody. Excess antibody is removed by washing as above and the plates developed by the addition of 100 μl substrate to each well.

The colour development reaction is stopped by the addition of 50 μl NaOH (3M) and the absorbance read at 405 and 690 nm.

### EXAMPLE 14

### Chemical Modification of PA, LF and/or EF

Amino acid-specific modification of cysteine residues or of amine groups on any amino acid residues are preferably targeted in the modified PA, LF and/or EF components of the present invention.

For example, titration of EF with varying concentrations of the sulphydryl-group reagent DTNB (5,5′-Dithiobis(2-nitrobenzoic acid)) irreversibly inhibits the adenylate cyclase activity of EF.

Similarly, treatment of PA and/or LF with the amine-specific reagent MLMS (mono(lactosylamido)mono(succinimidyl)suberate) irreversibly modulates the binding, activity of these two proteins. Titration with varying concentrations of MLMS may exert a range of effects upon the subsequent activities (ie. binding or enzyme activity) of these proteins.

### EXAMPLE 15

### Genetic Modification of PA, LF and/or EF

Site-directed mutagenesis of nucleotides residues encoding amino acid residues important for component (ie. PA, LF, or EF) binding may be used to modulate the activity of these three proteins.

For example, PA is the cellular-binding protein, which is cleaved at the cell-surface by the enzyme furin. The recognition site for the furin-cleavage event is RKKR. Site-directed mutagenesis of these amino acids would render the PA incapable of cleavage by furin and hence unable to bind to or internalise LF or EF.

Similarly, site-directed mutagenesis of residues 136-142 and 147-153 (VYYEIGK) of EF and LF, respectively, renders these proteins unable to bind to PA. Specifically, mutagenesis of the tyrosine residues, isoleucine or lysine residues is preferred to prevent binding to PA and hence formation of active toxins.

Site-directed mutagenesis is performed using mutagenic oligonucleotide primers followed by amplification of the desired region using the polymerase chain reaction. Mutagenised regions are then sequenced prior to reconstruction of the coding gene.

Alternatively, random mutations within the genes for the toxin components may be constructed by error-prone PCR. Four reactions are performed each using a nucleotide mix depleted for a different nucleotide. Each nucleotide mix contains a high concentration of deoxyinosine tri-phosphate (dITP), which would be incorporated at sites requiring the depleted nucleotide. As all four natural bases can pair with inosine, the probability that a mutation arises is 75% during the next PCR cycle.

### REFERENCES

K A Bradley, J Mogridge, M Mourez, R J Collier, J A T Young, Nature, 414, 225-229 (2001)
A M Friedlander, R Bhatnagar, S H Leppla, L Johnson, Y Singh, Infect Immun, 61, 245-52 (1993).

1-21. (canceled)

22. An antigenic pharmaceutical composition comprising 83 kDa Protective Antigen (PA) and Lethal Factor (LF), wherein PA lacks a functional binding site for a native PA cell receptor thereby preventing said PA from binding to said native cell receptor, and/or PA lacks a functional furin cleavage site thereby preventing said PA from in vivo cleavage by furin to form 63 kDa PA, and wherein said composition is substantially non-toxic to animal cells.

23. A composition according to claim 22, wherein the PA lacks said functional binding site for a native PA cell receptor, and wherein the PA lacks said functional furin cleavage site.

24. A composition according to claim 22, wherein said composition is substantially free of Lethal Toxin (LT) and Oedema Toxin (ET) activity.

25. A composition according to claim 22, wherein each of PA and LF lacks a functional binding site.

26. A composition according to claim 22, wherein PA lacks a functional binding site for LF thereby preventing said PA and LF from binding together via said binding site.

27. A composition according to claim 22, wherein LF lacks a functional binding site for PA thereby preventing said LF and PA from binding together via said binding site.

28. A composition according to claim 22, wherein said composition further comprises Oedema Factor (EF).

29. A composition according to claim 28, wherein said EF lacks a functional binding site for PA thereby preventing said EF and PA from binding together via said binding site.

30. A composition according to claim 22, wherein said LF has at least 30% metalloprotease activity when compared weight by weight with native LF.

31. A composition according to claim 28, wherein said EF has at least 30% adenylyl cyclase activity when compared weight by weight with native EF.

32. A composition according to claim 22, wherein said composition further comprises Sap 1 and/or EA1.

33. A composition according to claim 22, wherein PA and LF are each present at a concentration of 1-60 μg/ml.

34. The composition according to claim 33, wherein PA and LF are each present at a concentration of 2-20 μg/ml.

35. A composition according to claim 22, wherein the composition comprises PA and LF in weight ratios of 1:3 to 3:1.

36. The composition according to claim 35, wherein the composition comprises PA and LF in weight ratios of 1:1.5 to 1.5:1.

37. A recombinant method for preparing an antigenic composition according to claim 22, said method comprising:
expressing in a B. anthracis host cell a nucleic acid construct encoding said PA, and recovering the expressed PA;
expressing in a B. anthracis host cell a nucleic acid construct encoding said LF, and recovering the expressed LF; and
combining the PA and LF.

38. The method of claim 37, further comprising expressing in a B. anthracis host cell a nucleic acid construct encoding EF, and recovering the expressed EF; and
combining the EF with the PA and LF.

39. One or more DNA plasmid(s) that encode PA and LF as defined in claim 22; and wherein said plasmid(s) comprise a eukaryotic promoter that is operably linked to and, in use, drives expression of said PA and LF.

40. The DNA plasmid(s) of claim 39, further encoding EF.

41. One or more RNA vector(s) that encode PA and LF as defined in claim 22; and wherein the vector(s) comprise an integration site for a chromosome of a mammal host cell.

42. The RNA vector(s) of claim 41, further encoding EF.

43. A method of preventing or minimizing anthrax toxicity in a mammal, comprising administering to said mammal a composition according to claim 22, DNA plasmid(s) according to claim 39, or RNA vector(s) according to claim 41.

44. The method of claim 43, wherein said mammal is a human.

45. A method of preventing or minimizing anthrax toxicity in a mammal, comprising administering to said mammal a PA as defined in claim 22, wherein said PA is administered prior to, simultaneously with or subsequent to LF as defined in claim 22.

46. The method of claim 45, wherein said mammal is a human.
